(19) United States
(12) Patent Application Publication
(10) Pub. No.: US 2024/0325447 A1
(43) Pub. Date: Oct. 3, 2024

(54) GENERATION OF T CELL LINES WITH SPECIFICITY AGAINST ONE OR MORE NEOANTIGENS

(71) Applicant: Baylor College of Medicine, Houston, TX (US)

(72) Inventors: Ann Marie Leen, Houston, TX (US); Wingchi Leung, Houston, TX (US); Premal Lulla, Houston, TX (US)

(73) Assignee: Baylor College of Medicine, Houston, TX (US)

(21) Appl. No.: 18/698,646

(22) PCT Filed: Oct. 6, 2022
(86) PCT No.: PCT/US2022/077678

§ 371 Date: Apr. 4, 2024

Related U.S. Application Data
(60) Provisional application No. 63/253,675, filed on Oct. 8, 2021.

(51) Int. Cl. A61K 35/17; A61K 39/00; C07K 14/725; C12N 5/0783; C12N 15/86

(52) U.S. Cl. CPC A61K 35/17; A61K 39/4611; A61K 39/464401; C07K 14/7051; C12N 5/0636; C12N 15/86; C12N 2501/2306; C12N 2501/2307; C12N 2501/2312; C12N 2501/2315

(57) ABSTRACT

Embodiments of the disclosure include methods and compositions in which adoptive T cell therapy is generated by particular methods in which a population of the T cells are directed against one or more neoantigens of any kind. In specific embodiments, the T cells are produced following exposure of PBMCs to peptides that encompass one or more antigens, and the produced T cells are tested for their efficacy. Following identification of efficacious peptides, they are utilized to produce the adoptive T cell therapy against the neoantigen(s).

This application claims priority to U.S. Provisional Patent Application Ser. No. 63/253,675, filed on Oct. 8, 2021, which is incorporated by reference herein in its entirety.

### STATEMENT REGARDING FEDERALLY SPONSORED RESEARCH OR DEVELOPMENT

This invention was made with government support under P50 CA126752 awarded by National Institutes of Health-National Cancer Institute. The government has certain rights in the invention.

### TECHNICAL FIELD

Embodiments of the present disclosure generally concern at least the fields of cell biology, immunology, molecular biology, and medicine. In particular embodiments, the disclosure generally concerns immunotherapy for cancer.

### BACKGROUND

Neo-Antigen-Targeted T Cells for Detection, Prevention and/or Treatment of Cancer

One of the challenges of adoptive immunotherapy for non-viral cancers remains the identification of strongly immunogenic target antigens. Model tumor antigens are specifically and universally expressed on tumor cells in order to limit collateral damage, and ideally should be essential for the maintenance of the oncogenic phenotype of the tumor. While the majority of antigens do not meet these criteria, neo-antigens are a category that are uniquely present in cancer cells and hence represent potential targets for cancer therapies/diagnostics including adoptively transferred T cells. However, before targeting neo-antigens using an immunotherapeutic approach it is first important to determine whether the mutated peptides are immunogenic and to optimize cell culture conditions for neo-antigen-specific T cell production.

The present disclosure satisfies a long-felt need in the art to provide effective cancer-specific cell immunotherapy.

### BRIEF SUMMARY

The present disclosure relates to the production of T cells that target one or more neoantigens. In specific embodiments, the present disclosure provides methods to produce T cells targeting one or multiple neoantigens that can be used to treat individuals with cancer harboring the specific mutation(s) targeted by the neoantigen-specific T cells. In specific embodiments, the source of the neoantigen used to produce the neontigen-targeted T cells may be peptides encompassing the mutated sequence, used either alone or pooled with peptides encompassing a mutated sequence of another neoantigen to target multiple neoantigens. In certain embodiments, the neoantigen-specific T cells are cultured under specific conditions and/or environments, such as being cultured in tissue cultureware including multiwell substrates (e.g., 24-well tissue culture treated plates) and/or in vessels, such as vessels having gas permeable membranes. In specific embodiments, the culture medium comprises one or more specific cytokines, and the culturing may occur using one stimulation or multiple stimulations. When there are multiple stimulations, the specific conditions and/or environments may or may not be the same among the stimulations. For example, one or more subsequent stimulations following an initial stimulation may use a different tissue cultureware and/or different culture medium. In some cases, one or more subsequent stimulations following an initial stimulation comprises a different combination of cytokines, and one or more cytokines in the different stimulations may or may not overlap. In a specific case, the culture medium comprises IL-7, IL-12, IL-15, and IL-6 during an initial stimulation and comprises IL-7 and IL-15 and/or IL-2 at a subsequent stimulation, such as a second and/or further stimulation. In specific embodiments, the culture medium comprises 1, 2, 3, 4, 5, or more cytokines, and in other embodiments the culture medium consists essentially of 1, 2, 3, 4, or 5 or more cytokines, and in other embodiments, the culture medium consists of 1, 2, 3, 4, or 5 or more cytokines. In specific embodiments with respect to cytokines only, the culture medium comprises 1, 2, 3, 4, 5, or more cytokines, and in other embodiments the culture medium consists essentially of 1, 2, 3, 4, or 5 or more cytokines, and in other embodiments, the culture medium consists of 1, 2, 3, 4, or 5 or more cytokines; in such cases, the culture medium comprises one or more other components that are effective for cell stimulation and production.

For the generation of neoantigen-specific T cells, antigen presenting cells (APCs) such as dendritic cells (DCs) may be pulsed with one or more peptides, including one or more libraries of peptides, encompassing a mutated neoantigen peptide sequence. Alternatively, PBMCs can be stimulated directly with one or more peptides, including one or more libraries of peptides, encompassing a mutated neoantigen sequence.

In some embodiments, this disclosure provides for the targeting of cancers (hematological and/or solid tumors) or precancerous conditions (such as clonal hematopoiesis of indeterminate potential) that harbor the target neo-antigen(s) using T cells stimulated with peptides encompassing mutated sequences present in tumors including driver mutations (i.e., mutations that drive the malignant phenotype; “hot spot” mutations (i.e., mutations that are frequently detected across many different diseases/tumor types); and/or patient-specific neo-peptides identified by sequencing strategies including whole exome sequencing (WES) studies performed on fresh or banked tumor material. The disclosure concerns, at least in part, protocols to generate T cell lines either from individuals with cancer or healthy donors. These T cell lines with specificity for one or multiple neoantigens simultaneously may be generated using antigen presenting cells (e.g., DCs) pulsed with a mix of peptides encompassing mutant sequences. This approach allows for priming both CD4+ and CD8+ tumor-specific T cells. Targeting one or more neoantigens present within a given tumor maximizes the efficacy of adoptively transferred T cells and minimizes both the risk of off-tumor toxicities and the risk of tumor immune escape, in at least some embodiments. For in vitro expansion, in specific embodiments one can utilize an optimized cytokine cocktail to promote the selective expansion of tumor-reactive neospecific T cells. Resultant lines are tested for functionality in vitro such as by the production of one or multiple effector molecules including IFNγ, TNFα, and Granzyme B, for example when stimulated with neopeptides in ELIspot, Flurospot and intracellular cytokine assays, and specifically lysed peptide-pulsed, or naturally expressing, target cells in traditional Cr51 release assays.

Embodiments of the disclosure include methods of generating neoantigen-specific T cells that target at least one peptide comprising part or all of a neoantigen, said method comprising the steps of: (a) contacting a population of antigen presenting cells (APCs) with (1) an overlapping library of peptides spanning one mutated neoantigen sequence or with (2) an overlapping library of peptides wherein the peptides in the library collectively span multiple mutated neoantigen sequences, wherein for each library each mutated neoantigen sequence is optionally located at different positions among the individual peptides, thereby producing pepmix-loaded APCs; contacting peripheral blood mononuclear cells (PBMCs) from an individual with the pepmix-loaded APCs and performing at least one in vitro stimulation to produce a population of antigen-specific T cells that are capable of responding to at least one of the peptides; or (b) contacting PBMCs from an individual with cancer or a healthy individual with (1) an overlapping library of peptides spanning one mutated neoantigen sequence or with (2) an overlapping library of peptides wherein the peptides in the library collectively span multiple mutated neoantigen sequences, wherein for each library each mutated neoantigen sequence is optionally located at different positions among the individual peptides, and performing at least one in vitro stimulation to produce a population of antigen-specific T cells that are capable of responding to at least one of the peptides, wherein in (a) or (b) the in vitro stimulation comprises culturing in a medium supplemented with two or more cytokines selected from the group consisting of IL-7, IL-12, IL-15 and IL-6 to produce the neoantigen-specific T cells. In specific embodiments, the medium is supplemented with IL-7 and IL-15. In some methods, the steps further comprise the step of assaying for neo-epitope specificity of the neoantigen-specific T cells by assessing T cell activity against one or more particular peptides comprising the mutated neoantigen sequence in comparison to T cell activity against a correlative peptide comprising the corresponding wild type sequence. T cell activity may be assessed by production of IFNγ, TNFα direct cytotoxicity of neopeptide-expressing targets and/or Granzyme B. In specific cases, the T cell activity is assessed by ELIspot, Flurospot, single cell RNA sequencing, cytotoxicity assay, and/or one or more intracellular cytokine assays.

In some embodiments, the method further comprises the step of one or more additional in vitro stimulation steps, such as the one or more additional in vitro stimulation steps comprising culturing the cells in a medium comprising IL-7 and one or both of IL-15 and IL-2. The stimulation step may occur in a multiwell flask or a vessel with a gas permeable membrane. In specific aspects, the APCs are dendritic cells, B cells, monocytes, lymphoblastoid cell lines, gene modified or non-modified immortalized cell lines, K562 cells, or a mixture thereof. The mutated neoantigen sequence may comprise one or more modified amino acids, such as one or more amino acid substitutions, one or more amino acid deletions, one or more insertions, one or more inversions, or one or more translocations.

In particular embodiments, the PBMCs are from an individual that has cancer or a precancerous condition harboring one or more of the neoantigens. The PBMCs may be from an individual that is healthy and does not have cancer or a precancerous condition. In specific cases, a therapeutically effective amount of the neoantigen-specific T cells are administered to an individual that has cancer cells or pre-cancerous cells harboring one or more of the neoantigens. The neoantigen-specific T cells may be administered to the individual from which the PBMCs were obtained. The neoantigen-specific T cells may be administered to an individual other than the individual from which the PBMCs were obtained. In some cases, the individual to which the neoantigen-specific T cells are administered and the individual from which the PBMCs were obtained have 1, 2, 3, 4, 5, or more different HLA antigens. In certain embodiments, the neoantigen-specific T cells are comprised of CD8+ T cells and/or CD4+ T cells.

Particular embodiments of methods encompassed herein further comprise the step of identifying the α and/or β T-cell receptor (TCR) sequences of the neoantigen-specific T cells. The methods may further comprise the step of cloning the α and/or β T-cell receptor (TCR) sequences into a vector, including a viral vector (retroviral, lentiviral, adenoviral, or adeno-associated viral vector, for example) or a non-viral vector (plasmid or transposon, for example). Methods may further comprise the step of engineering T cells to express an engineered TCR comprising the α and/or β T-cell receptor (TCR) sequences, thereby producing engineered T cells. In some cases, the engineered TCR is engineered into the T cell receptor locus of the engineered T cells. In some cases, the constant region of the engineered TCR comprises a non-human constant region. The engineered TCR may or may not comprise one or more sequences that stabilize the α and β chains of the engineered TCR to facilitate suitable transgenic TCR pairing. The engineered TCR may comprise the addition of disulphide bonds, hydrophobic modifications, swapping constant domains of α and β chain, disrupting endogenous TCR expression, use of γδ constant domains, incorporation of CD3zeta to the TCR, use of single-chain TCR format, removal of one or more N-glycosylation sites, specific insertion of the TCR into the TCR locus of the target cells, use of TCR-deleted cells, or a combination thereof.

In some embodiments, the neoantigen-specific T cells and/or engineered T cells are administered by injection, such as intravenous. The cancer or precancerous condition may be hematological or may comprise solid tumors. The neoantigen may comprise a driver mutation, a hot spot mutation, or is specific to an individual.

In particular embodiments, the produced neoantigen-specific T cells are engineered to comprise one or more modifications, such as (a) disruption of one or more endogenous genes of the neoantigen-specific T cells; (b) one or more chimeric antigen receptors; (c) one or more non-natural antigen-specific T cell receptors other than the engineered receptor comprising the α and/or β T-cell receptor (TCR) sequences; (d) one or more cytokine receptors; (e) one or more chimeric cytokine receptors; (f) one or more cytokines; or (g) a combination thereof. In some embodiments, the non-natural T cell receptors of (c) are TCRs directed against a tumor-associated antigen or a virus. The neoantigen-specific T cells may be modified to be directed against a tumor-associated antigen and a virus. In specific embodiments, the antigen of (b) and/or the antigen of (c) is selected from the group consisting of PRAME, WT1, SSX2, MAGE, Survivin, MAGE-A4, SSX2, NY-ESO-1, Cyclin-A1, BCL-2, CML28, CML66, BRAP, Cyline B1, Cylin E, CYP1B1, hTERT, HOXA9, mesothelin, myeloperoxidase, MUC1, Proteinase 3, RGS5, RHAMM, MAGE, PASD1, RAGE-1, Lwis Y antigen, a-galactosylceramide, MOTCH, CD44v6, phosphopeptides, protein tyrosine phosphatase, CLL-1, CD19, CD20, BCMA, CD96, CD33, CD123, CD34, IL12RB1, B7H3, CD70, CD99, and a combination thereof.

Embodiments of the disclosure encompass any cells produced by any method encompassed herein, including any composition that comprises the cells. The composition may be comprised in a pharmaceutically acceptable excipient and/or one or more cryoprotectants.

In some embodiments, there is a method of diagnosing cancer or a precancerous condition in an individual, comprising the step of assaying for neoantigen-specific memory cells in a sample (peripheral blood, tumor material, lymph node, bone marrow, cerebrospinal fluid, or a mixture thereof, for example) from the individual, wherein when said neoantigen-specific memory cells are detected, said individual has cancer or a precancerous condition. The assaying step may occur in the absence of an enrichment step for the memory cells. or it may occur following an enrichment step for the memory cells. In specific embodiments, the enrichment step comprises stimulation with APCs specific for the neoantigen. The assaying step may further comprise assaying for secretion of one or more effector molecules (e.g., IL-2, TNF-α, IFNγ, and/or Granzyme B) when stimulated with one or more peptides that comprise the neoantigen. The assaying may comprise ELIspot, Flurospot, single cell RNA sequencing, cytotoxicity assay, and/or one or more intracellular cytokine assays.

In some embodiments, there is a method comprising the step of measuring neoantigen-specific T cells in a sample from an individual known to have or suspected of having cancer or from an individual known to have or suspected of having a precancerous condition. The method may further comprise the step of administering a therapeutically effective amount of a treatment to the individual when the neoantigen-specific T cells are measured in the sample from the individual. The treatment may comprise chemotherapy, immunotherapy (including cell therapy), radiation, surgery, hormone therapy, or a combination thereof. In specific cases, the cell therapy comprises T cells engineered to have T cell receptors directed to the neoantigen(s).

In certain embodiments, there is a method of treating an individual for cancer or a pre-cancerous condition, comprising the step of administering to the individual an effective amount of a treatment when the individual comprises neoantigen-specific memory cells. The method may further comprise the step of determining the presence of the neoantigen-specific memory cells. In some embodiments, there is a method for treating an individual with cancer or a precancerous condition comprising administering an effective amount of chemotherapy, immunotherapy, radiation, surgery, hormone therapy, or a combination thereof to the individual after the level of neoantigen-specific memory cells has been measured from a biological sample from the individual. In some embodiments, there is a method for evaluating an individual suspected of needing cancer therapy or prevention, comprising the step of measuring a sample from the individual for the presence of neoantigen-specific memory cells.

### DETAILED DESCRIPTION

### I. Examples of Definitions

The use of the word “a” or “an” when used in conjunction with the term “comprising” may mean “one,” but it is also consistent with the meaning of “one or more,” “at least one,” and “one or more than one.” As used in the specification and claims, the singular form “a”, “an”, and “the” include plural references unless the context clearly dictates otherwise.

The compositions and methods for their use can “comprise,” “consist essentially of,” or “consist of” any of the ingredients or steps disclosed throughout the specification. Throughout this specification, unless the context requires otherwise, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and will be understood to imply the inclusion of a stated step or element or group of steps or elements but not the exclusion of any other step or element or group of steps or elements. It is contemplated that embodiments described herein in the context of the term “comprising” may also be implemented in the context of the term “consisting of” or “consisting essentially of.” Compositions and methods “consisting essentially of” any of the ingredients or steps disclosed limits the scope of the claim to the specified materials or steps which do not materially affect the basic and novel characteristic of the claimed disclosure. The words “consisting of” (and any form of consisting of, such as “consist of” and “consists of”) means including, and limited to, whatever follows the phrase “consisting of.” Thus, the phrase “consisting of” indicates that the listed elements are required or mandatory, and that no other elements may be present.

The word “or” or the phrase “and/or” means “and” or “or”. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the inherent variation or standard deviation of error for the measurement or quantitation method being employed to determine the value.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. With respect to cells, the cells may be engineered because they express one or more heterologous genes (such as synthetic TCRs, receptors of any kind including antigen receptors, and/or cytokines) and/or the cells are engineered by having reduced expression of one or more endogenous genes, all of which case(s) the engineering is performed by the hand of man. With respect to an antigen receptor, including a TCR, the antigen receptor may be considered engineered because it comprises multiple components that are genetically recombined to be configured in a manner that is not found in nature, such as in the form of a fusion protein of components not found in nature so configured.

“Individual, “subject,” and “patient,” are used interchangeably herein and generally refers to an individual in need of treatment. The subject can be any animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having cancer. The subject may be undergoing or having undergone cancer treatment. The “subject” or “individual,” as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (e.g., children) and infants. The individual may be of any gender or race or ethnicity.

The terms “prevent”, “preventing, “prevention” and the like, as used herein, unless otherwise indicated, refers to inhibiting the onset of one or more symptoms or one or more complications, including inhibiting the onset of cancer metastasis. It includes the administration of any of the compositions, pharmaceutical compositions, or dosage forms described herein.

The terms “treat”, “treating”, “treatment” and the like, as used herein, unless otherwise indicated, refers to reversing, alleviating, or inhibiting the process, or delaying onset of the disease, disorder or condition to which such term applies, or one or more symptoms of such disease, disorder or condition and includes the administration of any of the compositions, pharmaceutical compositions, or dosage forms described herein, or eliminating the disease, condition, or disorder. In some instances, treatment is curative or ameliorating.

The terms “administering”, “administer”, “administration” and the like, as used herein, refer to any mode of transferring, delivering, introducing, or transporting a therapeutic agent to a subject in need of treatment with such an agent. Such modes include, but are not limited to, by injection, intraocular, oral, topical, intravenous, intraperitoneal, intramuscular, intradermal, intranasal, and subcutaneous administration.

As used herein, a “disruption” of a gene refers to the elimination or reduction of expression of one or more gene products encoded by the subject gene in a cell, compared to the level of expression of the gene product in the absence of the disruption. Exemplary gene products include mRNA and protein products encoded by the gene. Disruption in some cases is transient or reversible and in other cases is permanent. Disruption in some cases is of a functional or full length protein or mRNA, despite the fact that a truncated or non-functional product may be produced. In some embodiments herein, gene activity or function, as opposed to expression, is disrupted. Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

The term “heterologous” as used herein refers to being derived from a different cell type or a different species than the recipient. In specific cases, it refers to a gene or protein that is synthetic and/or not from a natural T cell. The term also refers to synthetically derived genes or gene constructs.

The term “neoantigen” as used herein refers to a protein or peptide arising from somatic mutation expressed only by cancer cells (or precancerous cells), including hematological malignancies and solid tumor cells.

The term “neoantigen-specific T-lymphocytes” or “neoantigen-specific T cell lines” or “neoantigen-specific T cells” or “neospecific T cells” are used interchangeably herein to refer to T cell lines that have specificity and potency against a cancer neoantigen or cancer neoantigens of interest or a cancer neo-epitope. As described herein, in at least some cases a neoantigen or two or several neoantigens are presented to native T cells in peripheral blood mononuclear cells and the native CD4 and CD8 T cell populations expand in response to said neoantigen(s). These cells can be generated from patients with cancer harboring these neoantigens or also from healthy individuals. The source of the responding T cells can be from the naïve T cell fraction or from the memory T cell pool. For example, a neoantigen-specific T cell line can recognize the neoantigen, thereby expanding the T cells specific for the neoantigen. In another example, a neoantigen-specific T cell line for a first neoantigen and a second neoantigen can recognize both the first and second neoantigens, thereby expanding the T cells specific for the first and second neoantigens. The methods of the disclosure allow for induction of CD8+ T cells, CD4+ T cells, or a combination of CD8+ T cells and CD4+ reactive T cells.

The term “neo-epitope” as used herein refers to a peptide harboring a mutated amino acid/nucleotide sequence capable of binding to class I and/or class II HLA molecules and being recognized by the T cell receptor of natural T cells or engineered cells including engineered T cells, NK cells, NKT cells, etc.

Other objects, feature and advantages of the present invention will become apparent from the following detailed description. It should be understood, however, that the detailed description and specific examples, while indicating specific embodiments of the invention, are given by way of illustration only, since various changes and modifications within the spirit and scope of the invention will become apparent to those skilled in the art from this detailed description.

The following discussion is directed to various embodiments of the disclosure. The term “invention” is not intended to refer to any particular embodiment or otherwise limit the scope of the disclosure. Although one or more of these embodiments may be particularly considered, the embodiments disclosed should not be interpreted, or otherwise used, as limiting the scope of the disclosure, including the claims. In addition, one skilled in the art will understand that the following description has broad applications, and the discussion of any embodiment is meant only to be exemplary of that embodiment, and not intended to intimate that the scope of the disclosure, including the claims, is limited to that embodiment.

Embodiments of the disclosure include methods and compositions that encompass use and production of T cells that target one or more neoantigens that can be adoptively transferred to individuals with cancer. The disclosure also provides for steps that may be taken prior to the delivery of the cells, such as useful steps for producing T cells that are more efficacious than T cells produced by other methods. In specific embodiments, the methods are enhanced compared to others at least because they identify whether one or more particular peptides associated with the neo-antigen(s) are immunogenic. Specific methods also utilize particular cell culture conditions for the production or stimulation of the neo-antigen-specific T cells, including at least in some cases using one or more certain cytokines in the cell culture medium.

### II. Neoantigens

The present disclosure provides cell immunotherapies that target somatic mutations expressed only by cancer cells or precancerous cells, otherwise known as neoantigens. In particular embodiments, the mutations are of any kind, including those that are the following: (1) driver mutations (i.e., mutations that cause cells to become cancer cells); (2) “hot spot” mutations (i.e., mutations that are frequently detected within one cancer type, or across many different diseases/tumor types and that occur more frequently than expected compared to a background frequency; and/or (3) a patient-specific mutations.

The nature of the mutations for the neoantigen may be of any kind of mutation, including a substitution, deletion, insertion, missense, nonsense, translocation, or frameshift, as examples. In some cases, the neoantigen comprises a neo-epitope that encompasses sequence surrounding the specific mutation. For example, there may be a single substitution at one amino acid, but the neoantigen comprises a neo-epitope of one or several amino acids on the N-terminal and/or C-terminal side of the single substitution.

The neoantigen may be associated with one or more types of cancers. For example, the neoantigen may be identified in a certain percentage of individuals with a first type of cancer and also associated in a lesser percentage of individuals with a second or third, etc., type of cancer. In some cases, the neoantigen is only identified in one particular type of cancer. Samples from an individual identified as having a certain type of cancer may be assayed for the presence of any type of neoantigen prior to determining whether or not the individual should be administered therapies encompassed herein.

The neoantigen may be associated with one or more particular genes, and those one or more genes may or may not be known to be prone to having cancer-causing mutations, such as those affiliated with a certain type of cancer. In certain embodiments, more than one neoantigen is known to be present in one or more genes for a particular type of cancer, and the individual may or may not be subject to methods of determining whether or not the more than one neotigen is present in the cells of the individual.

### III. Neoantigen Peptide Library

Methods of the disclosure utilize neopeptide libraries as a means to produce neoantigen-specific T cells having T-cell receptors (TCRs) that recognize at least one neopeptide. Neoantigen peptide libraries are a collection of peptides that represent a particular neoantigen. The neoantigen comprises at least one mutation, and the mutation may or may not be located at different positions within the collection of peptides. A given mutation may be successively staggered among a collection of peptides. Among a collection of peptides, a given mutation may be present on a first peptide at a first position, present on a second peptide at a second position that is adjacent to the first position, present on a third peptide at a third position that is adjacent to the second position, present on a fourth peptide at a fourth position that is adjacent to the third position, and so on. The mutation may be at or towards one end of the peptide, or the mutation may be generally centrally located within the peptide. In a specific case, a single peptide (e.g., 25-30 amino acids in length) is generated and the mutation is generally centrally located within the peptide. The library comprises a plurality of peptides at least some of which are non-identical, in specific cases, and such a library may be referred to as a pepmix. In some cases, a pepmix comprises two or more libraries, such as when each of the libraries comprise a collection of pepmixes that are directed to different neoantigens, and at least some of the peptides are non-identical within the libraries.

The peptides may be said to represent a particular neoantigen by each of the peptides comprising sequence of at least some of the neoantigen. Collectively, the pepmix may comprise the entire region of the neoantigen such that each peptide within a pepmix comprises the mutated region at a different location. In particular embodiments, each of the peptides comprise a specific mutation sequence of the neoantigen. Some of the peptides may comprise the mutation sequence at or towards an N-terminal end of the peptide, at or towards a C-terminal end of the peptide, between the center and the end of the peptide, or approximately in the center of the peptide. In specific cases, each mutated neoantigen sequence of a given library may be located at different positions among the individual peptides, although some of the peptides in a given library may be identical or substantially identical (e.g., greater than about 75, 80, 85, 90, or 95%) to one another.

Pepmixes utilized in the disclosure may or may not be from commercially available peptide libraries. The peptides may be 15 amino acids long, or about 15 amino acids long, and they may overlap one another, such as by 11 amino acids, or by about 11 amino acids, in certain aspects. In some cases, they may be generated synthetically. Examples include those from JPT Technologies (Springfield, VA) or Miltenyi Biotec (Auburn, CA). In particular embodiments, the neoantigen peptides are at least (about) 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and in specific embodiments there is overlap of at least (about) 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length, for example.

In some embodiments, the amino acids as used in the pepmixes have at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99, at least 99.9% purity, inclusive of all ranges and subranges therebetween. In some embodiments, the amino acids as used here in the pepmixes have at least 70% purity.

The mixture of different peptides may include any ratio of the different peptides, although in some embodiments each particular peptide is present at substantially the same numbers in the mixture as another particular peptide. The methods of preparing and producing pepmixes for multiviral cytotoxic T cells with broad specificity is described in US2018/0187152, which is incorporated by reference in its entirety.

### IV. Generation of Neoantigen-Specific T Cells

The disclosure encompasses methods of producing neoantigen-specific T cell therapy using methods that are enhanced compared to known methods. The methods generate neoantigen-specific T cells that target at least one neoantigen, including 1, 2, 3, 4, 5, or more neoantigens.

The methods allow for utilizing one or more libraries of neopeptides (peptide comprising part or all of a neoantigen) that span a given neoantigen or libraries of neopeptides each of which span a given neoantigen. The methods generally utilize steps of producing neoantigen-specific T cells, including with high specificity and cytotoxicity for the neoantigen without wild type sequence reactivity using optimized culturing methods, followed by (1) administering the produced neoantigen-specific T cells alone or in combination with other immune based therapies including cancer testis/tumor-associated antigen-specific T cells, virus-specific T cells, BiTEs, etc. to an individual in need thereof; or (2) generating engineered T cells (or any other effector immune cell including NK cells, NK-T cells, macrophages, etc.) having TCRs based on the sequence of the α and β chains of the TCR of the effective neoantigen-specific T cells; (3) utilizing the sequence of the α and β chains of the TCR of the effective neoantigen-specific T cells for diagnosing tumor cells expressing that mutation; or (4) manufacturing a molecule (like a chimeric antigen receptor or a bispecific T cell engager or coupled with a chemotherapeutic, etc.) that contains the neo-antigen binding TCR for immunotherapy of tumors.

The neoantigen-specific T cells may be produced by contacting a population of antigen presenting cells (APCs) (e.g., dendritic cells (DCs), B cells, monocytes, or a mixture thereof) with an overlapping library of peptides or libraries of peptides (either or which may be referred to as a pepmix). The peptides of a given library encompass a neoantigen (a mutated neoantigen sequence), and in specific embodiments the mutated sequence is located at different positions within each of the individual peptides. This allows for enhanced epitope processing and presentation of the immunogenic epitope peptide as compared to when all of the peptides in the library are substantially identical. In some cases, the APCs are exposed to multiple libraries at substantially the same time, and each of the libraries encompass peptides that span a different neoantigen from another neoantigen. Such contact between the APCs and the pepmix produces pepmix-loaded APCs.

Following this, the pepmix-loaded APCs are exposed to a sufficient amount of peripheral blood mononuclear cells (PBMC) from any individual (including an individual in need of the cell therapy of the method or another individual or individuals that are not in need of the therapy). Such exposure between the two cell populations occurs under suitable conditions in vitro to produce the desired neoantigen-specific T cells, and this step utilizes conditions that stimulate the production of the neoantigen-specific T cells that are capable of responding to at least one neopeptide.

In alternative embodiments, instead of exposing the pepmix to APCs to produce pepmix-loaded APCs and then exposing the pepmix-loaded APCs to PBMCs, one can exclude the APC exposure and instead expose the pepmix library or libraries to the PBMCs directly, given the presence of APCs in the PBMCs (e.g., B cells, monocytes, etc.). In both cases, one or more stimulation steps are utilized to produce neoantigen-specific T cells.

PBMCs may be isolated from an individual in need of treatment or diagnosis or from a healthy donor, including a donor that does not have any cancer or does not have a precancerous condition. The isolation of the PBMCs may occur by any suitable method, and, by way of example, it may be density centrifugation (gradient) (Ficoll-Paque), using cell preparation tubes (CPTs), or using SepMate tubes with freshly collected blood. The PBMC can comprise lymphocytes, monocytes, and dendritic cells. The lymphocytes can include T cells, B cells, and NK cells. In some embodiments, the PBMCs as used herein may be cultured or cryopreserved. In some embodiments, the process of culturing or cryopreserving the cells can include contacting the cells in culture with one or more neoantigens under suitable culture conditions to stimulate and expand neoantigen-specific T cells.

In some embodiments, the process of culturing or cryopreserving the cells can include contacting the cells in culture with one or more neo-epitopes related to one or more neoantigens under suitable culture conditions. In some embodiments, contacting the PBMCs with one or more neoantigens, or one or more neo-epitopes from one or more neoantigens, stimulates and expands a population of neoantigen-specific T cells from the respective donor's MNCs or PMBCs.

Any stimulation step employs particular medium and particular tissue culture vessels or substrates. The vessels may be flasks or tissue culture-treated or non tissue culture treated plates, and the vessels may or may not comprise a gas permeable membrane. In some cases, the stimulation occurs in the presence of one or more particular media components, including one or more particular cytokines. In some cases, a specific combination of cytokines is utilized, such as IL-7, IL-12, IL-15 and IL-6. There may or may not be a second or more stimulation step, and in such cases the media and/or vessel may or may not be the same as was utilized in the initial stimulation. A second or subsequent stimulation step may utilize a different combination of cytokines compared to a first stimulation step, and one or more cytokines in different stimulations may or may not overlap. In specific cases, a second or subsequent stimulation step utilizes media that comprises IL-7 and one or both of IL-15 and IL-2.

The neoantigen-specific T cell lines may have one or more certain characteristics, including comprising CD4+ T cells and/or CD8+ T cells. In a specific case, the neoantigen-specific T cell lines are predominantly CD8+ and comprise T cells derived from both central (CD62L+) and effector memory (CD62L−) populations. The neoantigen-specific T cells may produce one or more Th1 cytokines, including when stimulated by the neoantigen(s). Examples of the cytokines include IFN-gamma, IL-2, IL-10, and TNF-alpha/beta. In some embodiments, the neoantigen-specific T cells do not significantly lyse non-cancer cells in the recipient individual. The neoantigen-specific T cell lines may or may not be cryopreserved following production.

Once the neoantigen-specific T cells have been stimulated for expansion, they may be characterized or assessed, such as for neoantigen specificity and/or cytotoxicity. In specific embodiments, the neoantigen-specific T cells are assessed for secretion of appropriate effector molecules when stimulated with the appropriate neopeptide(s) but not the corresponding wild-type sequence peptide (or the wild-type peptides stimulates to a lesser degree). In specific cases, the effector cytokines include IL-2, TNF-α, IFNγ, and/or Granzyme B. Assays that may be employed include ELIspot, Flurospot and intracellular cytokine assays, and specifically killed peptide-pulsed autologous or HLA-matched or partially HLA-matched neoantigen-expressing target cells in traditional Cr51 release assays.

### Engineering of Cells

In some embodiments, the neoantigen-specific T cells produced by methods encompassed herein are utilized themselves as compositions for treatment for an individual in need thereof. In other cases, the neoantigen-specific T cells produced by methods encompassed herein are not themselves utilized as a therapeutic composition, but sequences of the TCRs of these produced cells are identified (for example, by means of TCR sequencing) and further utilized in therapeutic compositions, such as other cells. In some embodiments, the TCRs of the neoantigen-specific T cells are analyzed for their sequence, and the sequence of the TCRs is utilized in subsequently engineered T cells having these TCR (or similar, such as greater than 75, 80, 85, 90, or 95% identity) sequences. In specific cases, neoantigen-specific T cells known or demonstrated to be responsive to neopeptides, and not their corresponding wild-type peptides, will have their respective TCR sequences obtained. T cells then are engineered to express the neoantigen-specific TCR to produced engineered T cells (that may also be referred to as engineered neoantigen-specific T cells). The identified α and β TCR sequences may be cloned into an appropriate vector (viral or non-viral) and transduced or transfected into T or other immune (e.g., NK/NKT, γd T cells, etc.) cells. Surface expression may be confirmed, such as by flow cytometry. Specificity may be confirmed, such as by cytokine (IL-2, TNF-α, IFNγ, and/or Granzyme) release upon exposure to the neopeptide but not the corresponding wild-type sequence. Cytotoxicity may be confirmed, such as by killing of neopeptide-pulsed targets in a Cr⁵¹ release assay.

For the engineered cells expressing the neoantigen-specific TCR, measures may be taken to avoid mispairing with native TCR sequences in the T cells being engineered. For example, the constant regions for the engineered TCRs may be non-human, including murine. In addition, or alternatively, one may include sequences in the TCR that stabilize neo-TCR α and β chains to guarantee appropriate transgenic TCR pairing. Examples include the addition of disulphide bonds, hydrophobic modifications, swapping constant domains of α and β chain, disrupting endogenous TCR expression (e.g., via gene knockouts, siRNA, CRISPR, etc.), use of γδ constant domains, incorporation of CD3z to TCR, use of single chain TCR format, removal of N-glycosylation sites, specific insertion of the TCR into the TCR locus of the target cells, use of TCR deleted cells and others.

In any cells encompassed herein, including the neoantigen-specific T cells produced by methods encompassed herein and that are used for therapy, or cells that are engineered to express the TCRs of the neoantigen-specific cells produced by methods encompassed herein, may be modified by the hand of man to have one or more modifications. The modifications may be useful to increase the therapeutic efficacy of the cells, to increase the persistence of the cells, or both. The cells may be modified to express one or more molecules that are not endogenous to the cells, such as one or more synthetic receptors, such as chimeric antigen receptors, TCRs, chimeric cytokine receptors, cytokine receptors, and so forth. In some cases, the cells are modified to express one or cytokines. Such modifications may be modifications such that the non-endogenous molecule(s) is integrated into the cell genome (and this integration may or may not be directed to a specific locus), or the non-endogenous molecule(s) may be present on a vector (viral or non-viral) in the cell.

In particular embodiments, any cells are modified to express one or more antigen-specific receptors, including chimeric antigen receptors and/or antigen-specific TCRs (that are not the TCRs specific for the neoantigen). In such cases, the antigen may be a cancer antigen or a viral antigen or an antigen for another pathogen. The antigen-specific receptors may be tailored specifically to target an antigen associated with a cancer of an individual or tailored to target an antigen associated with a virus of an individual. In specific embodiments, the antigen associated with the cancer is PRAME, WT1, SSX2, MAGE, Survivin, MAGE-A4, SSX2, NY-ESO-1, Cyclin-A1, BCL-2, CML28, CML66, BRAP, Cyline B1, Cylin E, CYP1B1, hTERT, HOXA9, mesothelin, myeloperoxidase, MUC1, Proteinase 3, RGS5, RHAMM, MAGE, PASD1, RAGE-1, Lwis Y antigen, a-galactosylceramide, MOTCH, CD44v6, phosphopeptides, protein tyrosine phosphatase, CLL-1, CD19, CD20, BCMA, CD96, CD33, CD123, CD34, IL12RB1, B7H3, CD70, or CD99. In specific embodiments, the antigen associated with a virus is EBV, HHV6, CMV, Adenovirus, BK virus, RSV, Influenza, parainfluenza, rhinovirus, hMPV, PIV, SARS-CoV-2, coronavirus, Hepatitis B, Hepatitis C, HPV, or varicella zoster virus.

TCRs can be expressed into non-engineered cells with tumor antigen or virus specificities—e.g. PRAME, WT1, SSX2, MAGE, Survivin, Cyclin-A1, BCL-2, CML28, CML66, BRAP, Cyline B1, Cylin E, CYP1B1, hTERT, HOXA9, mesothelin, myeloperoxidase, MUC1, Proteinase 3, RGS5, RHAMM, MAGE, PASD1, RAGE-1, Lewis Y antigen, a-galactosylceramide, MOTCH, CD44v6, phosphopeptides, protein tyrosine phosphatase, CLL-1, CD19, CD20, BCMA, CD96, CD33, CD123, CD34, IL12RB1, B7H3, CD70, or CD99. In specific embodiments, the antigen associated with a virus is EBV, HHV6, CMV, Adenovirus, BK virus, RSV, Influenza, parainfluenza, rhinovirus, hMPV, PIV, SARS-CoV-2, coronavirus, Hepatitis B, Hepatitis C, HPV, or varicella zoster virus.

In some embodiments as an alternative to, or in addition to, the cells expressing one or more non-endogenous molecules, the cells may have disruption of one or more endogenous genes. The disrupted gene(s) may be of any kind and the disruption may be produced by any method, including CRISPR, for example. In some cases, the gene that is disrupted is the endogenous TCR of the T cell, PD-1, TIM-3, or other exhaustion markers.

### V. Methods of Treatment or Prevention

Embodiments of the disclosure encompass methods of treatment or prevention in which a therapeutically effective amount of cells produced by methods encompassed herein are administered to an individual in need thereof. In particular embodiments, there are methods of treating or preventing cancer or its precursor lesions in an individual comprising the step of administering by any suitable route an effective amount of neoantigen-specific T cells to an individual that has cancer or has a pre-cancerous condition or is at risk for cancer. The treatment methods will treat cancer in which the cancer has one or more neoantigens to which the neoantigen-specific T cells are directed. The prevention methods will prevent cancer from pre-cancerous cells (cells that are pre-destined to become cancerous over time, such as by genetics or following exposure to certain conditions) in which the pre-cancerous cells have one or more neoantigens to which the neoantigen-specific T cells are directed or prevent cancer in individuals at high risk of developing disease (for example, progression of clonal hematopoiesis or myelodysplasia to acute myeloid leukemia). In some cases, the methods of treatment or prevention allow for killing (including by lysing) of target cells that comprise the neoantigen(s) by contacting the target cells with an effective amount of the neoantigen-specific T cells, and in specific embodiments the contacting occurs in vivo in an individual that is the recipient of the cells.

In some cases, a therapeutic composition that comprises neoantigen-specific T cells are effective against more than one neoantigen because the population of neoantigen-specific T cells in the composition are specifically generated to recognize collectively the more than one antigen. In other cases, a therapeutic composition that comprises neoantigen-specific T cells are effective against more than one neoantigen because two or more independently generated cell lines that each are directed towards different neoantigens have been mixed or combined in the therapeutic composition. In alternative cases, the two or more independently generated cell lines are used in separate formulations for the same individual and may or may not be given at substantially the same time.

In specific embodiments, the individual is known to have cancer, is suspected of having cancer, or is at a higher risk for cancer when compared to the general population (a smoker, has a personal or family history, has exposure to the sun and/or environmental carcinogens, has a cancer-associated virus, such as HPV, is obese, is older, such as older than 40, etc.). Treatment with the produced cells may ameliorate one or more symptoms of cancer, may prevent cancer, may reduce the severity of one or more symptoms, may delay onset of the cancer, or may delay or reduce the extent of metastasis of the cancer.

In certain embodiments of the present disclosure, neoantigen-specific T cells are delivered to an individual in need thereof, such as an individual that has cancer. The neoantigen-specific T cells may be autologous with respect to the individual that has cancer, or the neoantigen-specific T cells may be allogeneic with respect to the individual that has cancer. In allogeneic embodiments, the neoantigen-specific T cells may be at least partially HLA matched as when utilized in an off-the-shelf approach. The cells then mediate direct anti-tumor effects and may also enhance the individual's immune system to attack the cancer cells. In particular embodiments, the treatment methods treat cancers in which at least one neoantigen is associated. In specific embodiments, the individual has cancer harboring one or more of the neoantigens for which the cells are targeted. In specific embodiments, the cancer is lung, breast, colon, hematological, bone, liver, kidney, brain, pancreas, uterine, skin, head and neck, ovarian, endometrial, testicular, stomach, gall bladder, spleen cancer, and so forth. Types of hematological cancers include leukemia (acute lymphocytic leukemia; acute myelogenous leukemia; chronic lymphocytic leukemia; chronic myeloid leukemia, etc.), lymphoma (Hodgkin lymphoma; non-Hodgkin lymphoma; chronic lymphocytic leukaemia (CLL); small lymphocytic lymphoma; etc.) and multiple myeloma (hyperdiploid or non-hyperdiploid). In specific cases, the cancer is primary, minimal residual disease, early cancer, advanced cancer, metastatic cancer, and/or relapsed refractory cancer, for example. The cancer may be of any stage, including stage I, II, III, or IV, for example.

The individual may utilize the treatment method of the disclosure as an initial treatment or after (and/or with) another treatment (e.g., chemotherapy, radiation, surgery, hormone therapy, and/or other types of immunotherapy). The immunotherapy methods may be tailored to the need of an individual with cancer based on the neoantigen, type, and/or stage of cancer, and in at least some cases the immunotherapy may be modified during the course of treatment for the individual. For example, if the cancer metastasizes, then the immunotherapy may change for a neoantigen associated with the metastasis or metastases. In other cases, the immunotherapy may be modified over the course of treatment for another neoantigen present in cancer cells of the individual.

In some embodiments, the present disclosure provides methods for immunotherapy comprising administering a therapeutically effective amount of the cells produced by methods of the present disclosure. In one embodiment, a medical disease or disorder is treated by transfer of cell populations produced by methods herein and that elicit an immune response. In certain embodiments of the present disclosure, cancer is treated by transfer of a cell population produced by methods of the disclosure and that elicits an immune response. Provided herein are methods for treating or delaying progression of cancer in an individual comprising administering to the individual an effective amount of neoantigen-specific cell therapy. The therapeutically effective amount of the produced cells for use in adoptive cell therapy is that amount that achieves a desired effect in a subject being treated. For instance, this can be the amount of neoantigen-specific T cells necessary to inhibit advancement or to cause regression of cancer.

In some cases, the individual is provided with one or more doses of the immune cells. In some embodiments, the composition as described herein is administered to the subject a plurality of times. In some embodiments, the composition as described herein is administered to the subject more than one time. In some embodiments, the composition as described herein is administered to the subject more than two times. In some embodiments, the composition as described herein is administered to the subject more than three times. In some embodiments, the composition as described herein is administered to the subject more than four times. In some embodiments, the composition as described herein is administered to the subject more than five times. In some embodiments, the composition as described herein is administered to the subject more than six times. In some embodiments, the composition as described herein is administered to the subject more than seven times. In some embodiments, the composition as described herein is administered to the subject more than eight times. In some embodiments, the composition as described herein is administered to the subject more than nine times. In some embodiments, the composition as described herein is administered to the subject more than ten times. In some embodiments, the composition as described herein is administered to the subject a number of times that are suitable for the subjects. In specific cases where the individual is provided with two or more doses of the immune cells, the duration between the administrations should be sufficient to allow time for propagation in the individual, and in specific embodiments the duration between doses is 1, 2, 3, 4, 5, 6, 7, or more days; or 1, 2, 3, 4, or more weeks; or 1, 2, 3, 4, 5, 6, 7, 8, 9, 10, 11, 12 or more months.

In specific embodiments, the cells are provided to an individual in a therapeutically effective amount (in a range from 10³ to 10¹⁰) that ameliorates at least one symptom related to neoantigen-expressing cancer cells in the individual. A therapeutically effective amount may be from 10³ to 10¹⁰, 10³ to 10⁹, 10³ to 10⁸, 10³ to 10⁷, 10³ to 10⁶, 10³ to 10⁵, 10³ to 10⁴, 10⁴ to 10¹⁰, 10⁴ to 10⁹, 10⁴ to 10⁸, 10⁴ to 10⁷, 10⁴ to 10⁶, 10⁴ to 10⁵, 10⁵ to 10¹⁰, 10⁵ to 10⁹, 10⁵ to 10⁸, 10⁵ to 10⁷, 10⁵ to 10⁶, 10⁶ to 10¹⁰, 10⁶ to 10⁹, 10⁶ to 10⁸, 10⁶ to 10⁷, 10⁷ to 10¹⁰, 10⁷ to 10⁹, 10⁷ to 10⁸, 10⁸ to 10¹⁰, 10⁸ to 10⁹, or 10⁹ to 10¹⁰ cells. Thus, in particular embodiments an individual having a particular neoantigen-positive cancer is provided once or multiple times a therapeutically effective amount of cells expressing T cells directed towards the neoantigen.

The produced cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of cells will be dependent on the subject being treated, the severity and type of the affliction, and the manner of administration. In some embodiments, doses that could be used in the treatment of human subjects range from at least 1×10³, at least 1×10⁴, 1×10⁴, at least 1×10⁵, at least 1×10⁶, at least 1×10⁷, at least 1×10⁸, at least 1×10⁹, or at least 1×10¹⁰ T cells/m². In a certain embodiment, the dose used in the treatment of human subjects ranges from about 1×10⁹ to about 1×10¹⁰ T cells/m². In additional embodiments, a therapeutically effective amount of T cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of T cells is readily determined by one of skill in the art based on the age, weight, sex, and physiological condition of the subject. Effective doses can be extrapolated from dose-response curves derived from in vitro or animal model test systems.

The present disclosure provides methods of treating or preventing cancer comprising administering to a subject in need thereof the compositions or the pharmaceutical compositions as described herein. In some embodiments, the T-lymphocytes between 1×10³ and 1×10⁹ T-lymphocytes/m², 1×10⁴ and 1×10⁸ T-lymphocytes/m², 1×10⁵ and 1×10⁷ T-lymphocytes/m², 1×10⁴ and 1×10⁸ T-lymphocytes/m², 1×10⁶ and 1×10⁹ T-lymphocytes/m², inclusive of all ranges and subranges therebetween. In some embodiments, the T-lymphocytes are administered to the subject. In some embodiments, the subject is immunocompromised.

Therapeutically effective amounts of the produced cells can be administered by a number of routes, including parenteral administration, for example, intravenous, intraperitoneal, intramuscular, intrasternal, intratumoral, intrathecal, intraventricular, through a reservoir, intraarticular injection, or infusion.

The present disclosure provides methods of lysing a target neoantigen-expressing cancer cell comprising contacting the target cell with the compositions or pharmaceutical compositions as described herein. In some embodiments, the contacting between the target neoantigen-expressing cancer cell and the compositions or pharmaceutical compositions occurs in vivo in a subject. In some embodiments, the contacting between the target cell and the compositions or pharmaceutical compositions occurs in vivo via administration of the neoantigen-specific T-cells to a subject. In some embodiments, the subject is a human.

The present disclosure provides pharmaceutical compositions comprising a polyclonal population of T-cells that recognize a plurality of neoantigens. In some embodiments, the present disclosure provides a polyclonal population of T-cells that recognize a plurality of neoantigens comprising at least one antigen from each of one or more genes.

In some embodiments, the present disclosure provides pharmaceutical compositions comprising the compositions as described herein formulated for intravenous delivery. In some embodiments, the composition as described herein is negative for bacteria. In some embodiments, the composition as described herein is negative for fungi. In some embodiments, the composition as described herein is negative for bacteria or fungi for at least 1 days, at least 2 days, at least 3 days, at least 4 days, at least 5 days, at least 6 days, at least 7 days, at least 8 days, at least 9 days, at least 10 days, in culture. In some embodiments, the composition as described herein is negative for bacteria or fungi for at least 7 days in culture.

In some embodiments, the pharmaceutical compositions formulated for intravenous delivery exhibit less than 1 EU/ml, less than 2 EU/ml, less than 3 EU/ml, less than 4 EU/ml, less than 5 EU/ml, less than 6 EU/ml, less than 7 EU/ml, less than 8 EU/ml, less than 9 EU/ml, less than 10 EU/ml of endotoxin. In some embodiments, the pharmaceutical compositions formulated for intravenous delivery are negative for mycoplasma.

The cells of the disclosure may be encompassed in a pharmaceutically acceptable carrier. As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

### VI. Methods of Diagnosis of Pre-Cancerous or Cancer Expressing Neoantigens

Embodiments of the disclosure include methods of diagnosing a pre-cancerous condition or cancer, wherein the pre-cancerous condition or cancer is associated with one or more neoantigens (e.g., a sample from an individual suspected of having a pre-cancerous condition or cancer has cells that express the neoantigen(s)). Other methods include measuring, assaying, detecting, or analyzying in a sample for one or more particular conditions associated with a pre-cancerous cells or pre-cancerous condition or cancer. In specific embodiments, the disclosure includes methods of treatment that are determined, or the need is determined, following methods of measuring, assaying, detecting, or analyzying in a sample for one or more particular conditions associated with a pre-cancerous condition or cancer.

Endogenous T cells in individuals with neoantigen-specificity can recognize neoantigens in pre-cancerous and cancerous lesions and develop into memory cells. These neoantigen-specific memory cells circulate in peripheral blood lymph nodes, tumor sites, bone marrow, and/or cerebrospinal fluid, and their detection can indicate the presence of the cancerous or pre-cancerous lesion in the individual, including for diagnosis. Thus, in specific embodiments, there are methods of measuring for the presence of neoantigen-specific memory cells in a sample from an individual suspected of having or known to have pre-cancerous cells or a pre-cancerous condition or cancer. In some cases, there are methods of detecting neoantigen-specific memory cells in an individual suspected of having or known to have pre-cancerous cells or a re-cancerous condition or cancer. In some cases, there are methods of analyzing or assaying a sample for the presence of neoantigen-specific memory cells in a sample from an individual suspected of having or known to have pre-cancerous cells or a pre-cancerous condition or cancer. In specific embodiments, upon identification of neoantigen-specific memory cells in a sample from an individual suspected of having or known to have pre-cancerous cells or a pre-cancerous condition or cancer, the individual is appropriately treated with any suitable kind of treatment (or prevention when the individual has pre-cancerous cells or a pre-cancerous condition). The treatment/prevention may including neoantigen-specific T cells and/or one or more other cancer treatments, such as chemotherapy, immunotherapy, hormone therapy, radiation, surgery, etc.

The disclosure includes methods of detecting neoantigen-specific memory cells directed against one or more neoantigens in an individual suspected of having or known to have pre-cancerous cells or a pre-cancerous condition or cancer, comprising performing a step for detecting the neoantigen-specific memory cells in a sample from the individual and, upon detection, treating the individual with a therapy for the cancer or a preventative for the pre-cancerous condition. Also disclosed herein are methods for treating cancer, or preventing onset of cancer in an individual with pre-cancerous cells or a pre-cancerous condition, the method comprising detecting neoantigen-specific T cells in a sample from the individual and administering to the individual an effective amount of neoantigen-specific T cells and/or one or more other cancer treatments, such as chemotherapy, immunotherapy, hormone therapy, radiation, surgery, etc. Methods of the disclosure include methods of treating cancer or treating a pre-cancerous condition or preventing cancer in an individual with a pre-cancerous condition, comprising the step of administering to the individual an effective amount of neoantigen-specific T cells and/or one or more other cancer treatments, such as chemotherapy, immunotherapy, hormone therapy, radiation, surgery, after determining that the individual has neoantigen-specific memory T cells in a sample from the individual.

Thus, embodiments of the disclosure encompass methods of diagnosing pre-cancer or cancer by identifying neoantigen specificity in memory cells. The memory cells can be assessed in the absence of an enrichment step in which the memory cells are first contacted with APCs that have been contacted with one or a plurality of pepmix libraries that encompass the neoantigen(s). In other cases, the memory cells can be assessed following an enrichment step in which the memory cells are stimulated with APCs that have been contacted with one or a plurality of pepmix libraries that encompass the neoantigen(s).

The memory T cells (whether or not they have undergone expansion) may be assayed for specificity. In some cases, the memory T cells may be exposed to at least one pepmix library that encompasses one or more neoantigens. Specificity of the memory T cells may be determined by secretion of one or more appropriate effector molecules when stimulated with the appropriate neopeptide(s), such as IL-2, TNF-α, IFNγ, and/or Granzyme B. Assays that may be employed include ELIspot, Flurospot and intracellular cytokine assays, and specifically killed peptide-pulsed autologous or HLA-matched neoantigen-expressing target cells in traditional Cr51 release assays. Specificities can also be determined by using multimers (HLA and neopeptide conjugate molecules that are used to isolate specific TCRs) or identification of TCR sequences with known reactivity to neopeptides.

In some embodiments of the present methods, the methods further comprise obtaining the sample from the individual. The sample may or may not be stored prior to the present methods steps.

What is claimed:

1. A method of generating neoantigen-specific T cells that target at least one peptide comprising part or all of a neoantigen, said method comprising the steps of:
(a) contacting a population of antigen presenting cells (APCs) with (1) an overlapping library of peptides spanning one mutated neoantigen sequence or with (2) an overlapping library of peptides wherein the peptides in the library collectively span multiple mutated neoantigen sequences, wherein for each library each mutated neoantigen sequence is optionally located at different positions among the individual peptides, thereby producing pepmix-loaded APCs;
contacting peripheral blood mononuclear cells (PBMCs) from an individual with the pepmix-loaded APCs and performing at least one in vitro stimulation to produce a population of antigen-specific T cells that are capable of responding to at least one of the peptides; or
(b) contacting PBMCs from an individual with cancer or a healthy individual with (1) an overlapping library of peptides spanning one mutated neoantigen sequence or with (2) an overlapping library of peptides wherein the peptides in the library collectively span multiple mutated neoantigen sequences, wherein for each library each mutated neoantigen sequence is optionally located at different positions among the individual peptides, and
performing at least one in vitro stimulation to produce a population of antigen-specific T cells that are capable of responding to at least one of the peptides,
wherein in (a) or (b) the in vitro stimulation comprises culturing in a medium supplemented with two or more cytokines selected from the group consisting of IL-7, IL-12, IL-15 and IL-6 to produce the neoantigen-specific T cells.

2. The method of claim 1, wherein the medium is supplemented with IL-7 and IL-15.

3. The method of claim 1 or 2, further comprising the step of assaying for neo-epitope specificity of the neoantigen-specific T cells by assessing T cell activity against one or more particular peptides comprising the mutated neoantigen sequence in comparison to T cell activity against a correlative peptide comprising the corresponding wild type sequence.

4. The method of claim 3, wherein the T cell activity is assessed by production of IFNγ, TNFα, direct cytotoxicity of neopeptide-expressing targets and/or Granzyme B

5. The method of claim 3 or 4, wherein the T cell activity is assessed by ELIspot, Flurospot, single cell RNA sequencing, cytotoxicity assay, and/or one or more intracellular cytokine assays.

6. The method of any one of claims 1-5, further comprising the step of one or more additional in vitro stimulation steps.

7. The method of claim 6, wherein the one or more additional in vitro stimulation steps comprises culturing the cells in a medium comprising IL-7 and one or both of IL-15 and IL-2.

8. The method of any one of claims 1-7, wherein the stimulation step occurs in a multiwell flask or a vessel with a gas permeable membrane.

9. The method of any one of claims 1-8, wherein the APCs are dendritic cells, B cells, monocytes, lymphoblastoid cell lines, gene modified or non-modified immortalized cell lines, K562 cells, or a mixture thereof.

10. The method of any one of claims 1-9, wherein the mutated neoantigen sequence comprises one or more modified amino acids.

11. The method of claim 10, wherein the one or more modified amino acids comprises one or more amino acid substitutions, one or more amino acid deletions, one or more insertions, one or more inversions, or one or more translocations.

12. The method of any one of claims 1-11, wherein the PBMCs are from an individual that has cancer or a precancerous condition harboring one or more of the neoantigens.

13. The method of any one of claims 1-12, wherein the PBMCs are from an individual that is healthy and does not have cancer or a precancerous condition.

14. The method of any one of claims 1-13, wherein a therapeutically effective amount of the neoantigen-specific T cells are administered to an individual that has cancer cells or pre-cancerous cells harboring one or more of the neoantigens.

15. The method of claim 14, wherein the neoantigen-specific T cells are administered to the individual from which the PBMCs were obtained.

16. The method of claim 14, wherein the neoantigen-specific T cells are administered to an individual other than the individual from which the PBMCs were obtained.

17. The method of claim 15, wherein the individual to which the neoantigen-specific T cells are administered and the individual from which the PBMCs were obtained have 1, 2, 3, 4, 5, or more different HLA antigens.

18. The method of any one of claims 1-17, wherein the neoantigen-specific T cells are comprised of CD8+ T cells and/or CD4+ T cells.

19. The method of any one of claims 1-18, further comprising the step of identifying the α and/or β T-cell receptor (TCR) sequences of the neoantigen-specific T cells.

20. The method of claim 19, further comprising the step of cloning the α and/or β T-cell receptor (TCR) sequences into a vector.

21. The method of claim 20, wherein the vector is a viral vector or a non-viral vector.

22. The method of claim 21, wherein the viral vector is retroviral, lentiviral, adenoviral, or adeno-associated viral vector.

23. The method of claim 21, wherein the non-viral vector is a plasmid or transposon.

24. The method of any one of claims 18-23, further comprising the step of engineering T cells to express an engineered TCR comprising the α and/or β T-cell receptor (TCR) sequences, thereby producing engineered T cells.

25. The method of claim 24, wherein the engineered TCR is engineered into the T cell receptor locus of the engineered T cells.

26. The method of claim 24 or 25, wherein the constant region of the engineered TCR comprises a non-human constant region.

27. The method of any one of claims 24-26, wherein the engineered TCR comprises one or more sequences that stabilize the α and β chains of the engineered TCR to facilitate suitable transgenic TCR pairing.

28. The method of claim 27, wherein the engineered TCR comprises the addition of disulphide bonds, hydrophobic modifications, swapping constant domains of α and β chain, disrupting endogenous TCR expression, use of γδ constant domains, incorporation of CD3zeta to the TCR, use of single-chain TCR format, removal of one or more N-glycosylation sites, specific insertion of the TCR into the TCR locus of the target cells, use of TCR-deleted cells, or a combination thereof.

29. The method of any one of claims 1-28, wherein the neoantigen-specific T cells and/or engineered T cells are administered by injection.

30. The method of claim 29, wherein the injection is intravenous.

31. The method of any one of claims 14-30, wherein the cancer or precancerous condition is hematological or comprises solid tumors.

32. The method of any one of claims 1-31, wherein the neoantigen comprises a driver mutation, a hot spot mutation, or is specific to an individual.

33. The method of any one of claims 1-32, wherein the produced neoantigen-specific T cells are engineered to comprise one or more modifications.

34. The method of claim 33, wherein the one or more modifications comprises:
(a) disruption of one or more endogenous genes of the neoantigen-specific T cells;
(b) one or more chimeric antigen receptors;
(c) one or more non-natural antigen-specific T cell receptors other than the engineered receptor comprising the α and/or β T-cell receptor (TCR) sequences;
(d) one or more cytokine receptors;
(e) one or more chimeric cytokine receptors;
(f) one or more cytokines; or
(g) a combination thereof.

35. The method of claim 34, wherein the non-natural T cell receptors of (c) are TCRs directed against a tumor-associated antigen or a virus.

36. The method of any one of claims 33-35, wherein the neoantigen-specific T cells are modified to be directed against a tumor-associated antigen and a virus.

37. The method of claim 34-36, wherein the antigen of (b) and/or the antigen of (c) is selected from the group consisting of PRAME, WT1, SSX2, MAGE, Survivin, MAGE-A4, SSX2, NY-ESO-1, Cyclin-A1, BCL-2, CML28, CML66, BRAP, Cyline B1, Cylin E, CYP1B1, hTERT, HOXA9, mesothelin, myeloperoxidase, MUC1, Proteinase 3, RGS5, RHAMM, MAGE, PASD1, RAGE-1, Lwis Y antigen, a-galactosylceramide, MOTCH, CD44v6, phosphopeptides, protein tyrosine phosphatase, CLL-1, CD19, CD20, BCMA, CD96, CD33, CD123, CD34, IL12RB1, B7H3, CD70, CD99, and a combination thereof.

38. Cells produced by the method of any one of claims 1-37.

39. A composition comprising the cells of claim 38.

40. The composition of claim 39, comprised in a pharmaceutically acceptable excipient.

41. The composition of claim 39 or 40, comprised in one or more cryoprotectants.

42. A method of diagnosing cancer or a precancerous condition in an individual, comprising the step of assaying for neoantigen-specific memory cells in a sample from the individual, wherein when said neoantigen-specific memory cells are detected, said individual has cancer or a precancerous condition.

43. The method of claim 42, wherein said sample comprises peripheral blood, tumor material, lymph node, bone marrow, cerebrospinal fluid, or a mixture thereof.

44. The method of claim 42 or 43, wherein said assaying step occurs in the absence of an enrichment step for the memory cells.

45. The method of claim 42 or 43, wherein said assaying step occurs following an enrichment step for the memory cells.

46. The method of claim 45, wherein the enrichment step comprises stimulation with APCs specific for the neoantigen.

47. The method of any one of claims 42-46, wherein said assaying step further comprises assaying for secretion of one or more effector molecules when stimulated with one or more peptides that comprise the neoantigen.

48. The method of claim 47, wherein the effector molecules comprise IL-2, TNF-α, IFNγ, and/or Granzyme B.

49. The method of any one of claims 42-48, wherein the assaying comprises ELIspot, Flurospot, single cell RNA sequencing, cytotoxicity assay, and/or one or more intracellular cytokine assays.

50. A method, comprising the step of measuring neoantigen-specific T cells in a sample from an individual known to have or suspected of having cancer or from an individual known to have or suspected of having a precancerous condition.

51. The method of claim 50, further comprising the step of administering a therapeutically effective amount of a treatment to the individual when the neoantigen-specific T cells are measured in the sample from the individual.

52. The method of claim 51, wherein the treatment comprises chemotherapy, immunotherapy, radiation, surgery, hormone therapy, or a combination thereof.

53. The method of claim 52, wherein the immunotherapy comprises cell therapy.

54. The method of claim 53, wherein the cell therapy comprises T cells engineered to have T cell receptors directed to the neoantigen(s).

55. A method of treating an individual for cancer or a pre-cancerous condition, comprising the step of administering to the individual an effective amount of a treatment when the individual comprises neoantigen-specific memory cells.

56. The method of claim 55, further comprising the step of determining the presence of the neoantigen-specific memory cells.

57. A method for treating an individual with cancer or a precancerous condition comprising administering an effective amount of chemotherapy, immunotherapy, radiation, surgery, hormone therapy, or a combination thereof to the individual after the level of neoantigen-specific memory cells has been measured from a biological sample from the individual.

58. A method for evaluating an individual suspected of needing cancer therapy or prevention, comprising the step of measuring a sample from the individual for the presence of neoantigen-specific memory cells.
